(19) United States
(12) Patent Application Publication
(10) Pub. No.: US 2005/0075561 A1
(43) Pub. Date: Apr. 7, 2005

(54) Method and apparatus for indicating an encountered obstacle during insertion of a medical device

(75) Inventor: Robert N. Golden, Kirkland, WA (US)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Lucent Medical Systems, Inc., Kirkland, WA (US)

(21) Appl. No.: 10/676,678

(22) Filed: Oct. 1, 2003

(51) Int. Cl. A61B 5/05

(52) U.S. Cl. 600/424

(57) ABSTRACT

A technique for detecting and indicating an internal anatomical obstacle encountered during insertion of a medical device into the body of a patient, comprising an elongated member such as a tube, catheter, guidewire, or other device, having a location indicating element, such as a permanent magnet, flexibly coupled to its distal end, and an external detector that tracks and displays the location and orientation of the location indicating element. The flexible coupling has sufficient stiffness to maintain the orientation of the location indicating element against the forces from both gravity and flowing blood within a patient's vasculature, but allows the location indicating element to change orientation if it encounters an obstacle during insertion. The medical caregiver monitors the detector's display and determines encounters with obstacles by observing changes in the orientation of the location indicating element.

### BACKGROUND OF THE INVENTION

1. Field of the Invention

The present invention is directed generally to medical devices and, more particularly, to a method and apparatus for detecting and indicating encounters with internal, anatomical obstacles during the insertion of a medical device in the body of a patient.

2. Description of the Related Art

Many medical procedures require the placement of a catheter, tube, guidewire, or other device into the body of a patient, and these devices may be inserted into and/or through veins, arteries, the mouth, nose, or other openings. The ultimate destination of the distal tip of the inserted medical device may be at some distance from its insertion point. For example, a catheter may be inserted into a patient's femoral vein at the groin, with the goal of placing the catheter's distal end at the midpoint of the patient's superior vena cava, above the right atrium of the heart.

Safe placement of such devices is often facilitated with the use of radiographic imaging, such as fluoroscopy. This imaging necessitates the use of expensive equipment, facilities, and personnel, and radiographic imaging also carries ionizing radiation risks to both the patient and caregivers.

Alternatives to radiographic imaging exist, which instead track the location of the inserted medical device via oscillating electromagnetic or static magnetic field sensing. For example, U.S. Pat. No. 5,568,809 discloses an invention that uses external magnetic field generators and a catheter containing a set of receiving antennas to localize and track the catheter during a cardiac procedure. U.S. Pat. Nos. 5,879,297; 6,129,668; 6,216,028; and 6,263,230 disclose inventions which track the three dimensional (3D) location and orientation of a permanent magnet attached to a medical device, and displays that information in real-time to the caregiver. However, these inventions do not image a patient's internal anatomy, and the caregiver relies upon the correspondence between external anatomical landmarks and the magnet's displayed location underneath those landmarks to correctly place the medical device.

Now, there is also often the need during the medical device insertions discussed above to determine not only the device's current location, but if the distal tip of the device has run into an obstacle. For example, a cardiac catheter may be unintentionally pushed up against the wall of the right atrium, which could lead to cardiac injury or even puncture, resulting in death. Although radiographic imaging can show this dangerous situation to the caregiver, the non-radiographic alternatives, at present, do not.

Therefore, it can be appreciated that there is a need for a device that will provide non-radiographic tracking systems a way to display obstacles during device insertion. The present invention provides this, and other advantages, as will be apparent from the following detailed description and accompanying figures.

### BRIEF SUMMARY OF THE INVENTION

The present disclosure is directed to techniques for detecting and indicating an encounter with an internal, anatomical obstacle while inserting a medical device. In an exemplary embodiment, the invention comprises a medical device designed for insertion into the body and includes an elongated member having a proximal end and distal end. A location indicating element is flexibly coupled to the distal end of the elongated member and is capable of being tracked from a location external to the body wherein an encounter with an obstacle causes a change in the orientation of the location indicating element.

In one embodiment, the location indicating element may be flexibly coupled to the elongated member using an elastic polymer. In another embodiment, the device further comprises a chamber flexibly coupled to the distal end of the tube and having a predetermined orientation with the tube wherein the location indicating element is contained within the chamber and wherein an encounter with the obstacle causes a change in the orientation of the chamber.

In one embodiment, the chamber is coupled to the distal end of the elongated member using a flexible joint member. In an exemplary embodiment, the flexible joint member has sufficient stiffness to maintain the orientation of the chamber under the influence of both gravity and the forces from flowing blood within a patient's vasculature.

The elongated member may be implemented in the form of many different insertable medical devices, such as a tube, catheter, guidewire or other medical device.

The flexible joint member may be provided in a variety of different embodiments. In one embodiment, the flexible joint member may be a solid rubber member or a hollow rubber member. The flexible joint member may also be provided in the form of a flexible metal member, or an elastic polymer member.

Similarly, the location indicating element may be provided in a variety of different embodiments. In one embodiment, the location indicating element may comprise one permanent magnet or a series of permanent magnets whose magnetic orientation is aligned such that the magnetic fields are additively combined. In an alternative embodiment, the location indicating element may be an electromagnet, a radio frequency coil, an antenna, a train relief sensor, or the like.

### BRIEF DESCRIPTION OF THE SEVERAL VIEWS OF THE DRAWING(S)

FIG. 1 is a drawing illustrating an exemplary embodiment of a device to detect the encounter with an obstacle.

FIG. 2 illustrates the use of the detector when an obstacle is encountered.

FIG. 3 illustrates the orientation of a location indicating element and external display thereof with no obstacle present.

FIG. 4 illustrates the orientation of a location indicating element and external display thereof with an obstacle present.

FIGS. 5A-5E are illustrations of alternative embodiments of the device.

### DETAILED DESCRIPTION OF THE INVENTION

As will be discussed in greater detail herein, a method and apparatus disclosed herein can indicate internal, anatomical obstacles encountered during medical device insertion by allowing the inserted medical device's tracked element, when attached to the distal end of the medical device, to easily bend at an angle to the direction of travel.

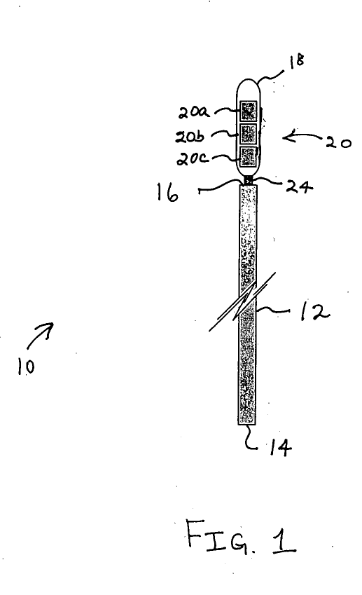

An exemplary embodiment of the present invention is illustrated in a system 10 illustrated in FIG. 1. The system 10 includes a medical elongated member 12 having a proximal end portion 14 and a distal end portion 16. The distal end portion 16 is designed for insertion into the body. For example, the elongated member 12 may be designed as a cardiac catheter with the distal end portion 16 being inserted into a desired location within the heart.

A vessel or chamber 18 is flexibly coupled to the distal end portion 16 of the elongated member 12. Contained within the chamber 18 is a location indicating element 20. In one embodiment, the location indicating element 20 may comprise multiple elements 20a-20c.

The location indicating element 20 may be implemented in a variety of embodiments. In an exemplary embodiment, the location indicating element 20 comprises a permanent magnet. The permanent magnet is capable of being detected from outside the body using known technology, such as that described in the previously referenced patents. The implementation of the location indicating element 20 as a permanent magnet advantageously provides an indication of the orientation of the magnet and thus the distal end portion 16 of the elongated member 12. The magnetic dipole give an indication of the location and orientation. The permanent magnet implementation of the location indicating element 20 requires no external power, or indeed, any power at all.

Alternatively, the location indicating element 20 may be implemented using a coil or antenna, such as used in an oscillating electromagnetic sensing device, radio frequency device, a strain relief sensor, piezoelectric device, or the like. The present invention is not limited by the specific implementation of the location indicating element 20.

When inserted into the body, the location indicating element 20 has a predetermined orientation with respect to the elongated member 12, as illustrated in FIG. 1. During a normal insertion process, that orientation is generally maintained without change. If the elongated member 12 encounters an obstacle, the obstacle causes a change in the orientation of the location indicating element 20, as illustrated in FIG. 2. The change in orientation is detected from outside the body, using known detector technology, as described above, to thereby indicate to the caregiver that the medical elongated member 12 has encountered an obstacle.

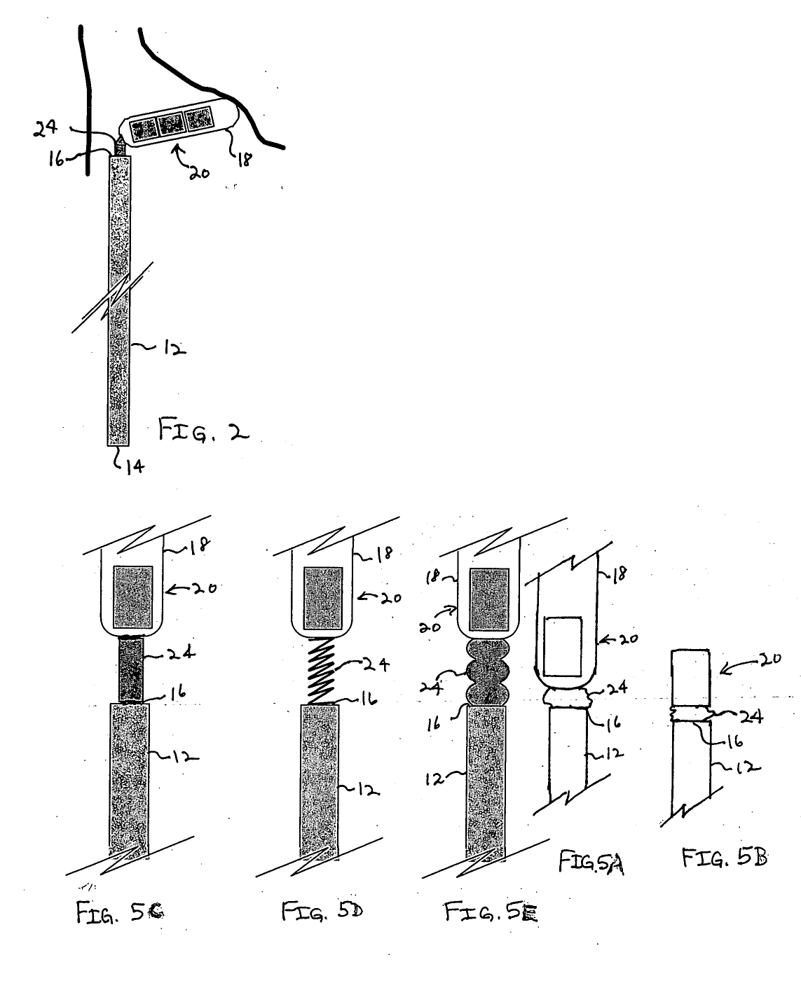

FIG. 3 illustrates a detector system 30 capable of determining the location and/or orientation of the location indicating element 20 from a location external to the body. The location and/or orientation can be shown on an external display 32. In the example of FIG. 3, an icon or arrow 34 shows the location and/or orientation of the location indicating element 20. A visible trail 36 can be shown on the display 32 to visually indicate preview and/or orientation of the location indicating element 20. FIG. 3 also illustrates the orientation of the location indicating element 20 and the corresponding external display 32 of the orientation when no obstacle is present. If an obstacle is encountered, the orientation of the location indicating element 20 changes with respect to the elongated member 12, as shown in FIG. 4. The external display 32 also changes to indicate the change in orientation to thereby alert the caregiver that an obstacle has been encountered. The caregiver may withdraw the elongated member 12 a short distance until the orientation is correct, as shown in FIG. 3, and proceed with the insertion of the elongated member 12.

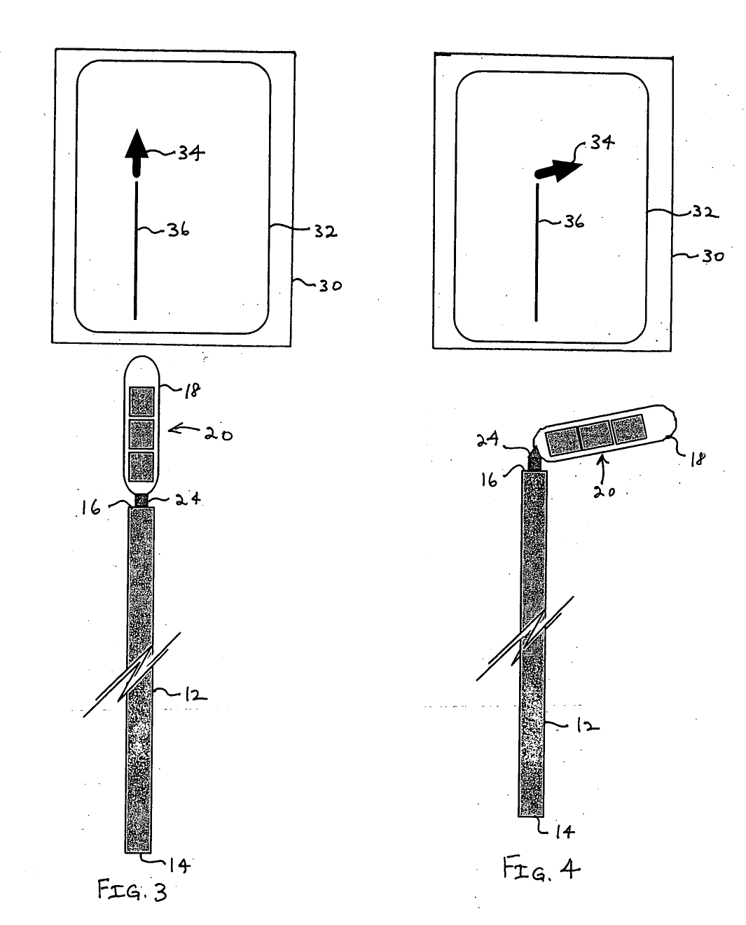

A number of alternative embodiments for the location-indicating element 20 have been discussed above. In addition, a number of alternative embodiments may be provided for attaching the location indicating element 20 to the distal end portion 16 of the elongated member 12. The location indicating element 20 may be attached directly to the distal end portion 16 as illustrated in FIG. 5B. The location indicating element 20 may be attached to the distal end portion 16 using known flexible compounds, such as silicone, elastic polymers, rubber, or the like.

FIG. 5A illustrates an alternative embodiment in which the location indicating element 20 may be located within the chamber 18. The chamber 18 is, in turn, coupled directed to the distal end portion 16 of the elongated member 12 using known flexible compounds, such as those discussed above.

In yet another alternative embodiment, the chamber 18 is coupled to the distal end portion 16 of the elongated member 12 using a flexible joint member 24. The flexible joint member 24 has sufficient stiffness that the orientation of the chamber 18 is maintained under the influence of both gravity and the forces from flowing blood within a patient's vasculature, but will flex when the chamber 18 encounters an obstacle, as illustrated in FIG. 2.

The flexible joint member 24 may be implemented in a variety of different embodiments. The flexible joint member 24 may have a cylindrical shape and be solid (i.e., a rod) or hollow (i.e., a tube), as shown in FIG. 5C. Alternatively, the flexible joint member 24 may be implemented as a spring, shown in FIG. 5D or a jointed connector, as shown in FIG. 5E. In addition to the variety of shapes, the flexible joint member 24 may be implemented by a variety of different materials. For example, the flexible joint member 24 may be a solid rubber member, a hollow rubber member (i.e., a tube), a flexible metal member, such as a spring, an elastic polymer, or the like. The selection of compounds for the implementation of the flexible joint member 24 must take into account the environment in which the elongated member 12 will exist. That is, the chamber 18 and flexible joint member 24 will both be placed inside the body. Accordingly, those elements must be selected so as not to break down within the body. For example, the metal implementation of the flexible joint member 24 may use stainless steel in the form of a spring, a metal rod, or the like. The present invention is not limited by the specific element or elements used to implement the chamber 18 or the flexible joint member 24.

In summary, the location indicating element 20 might be a coil or antenna in the case of oscillating electromagnetic sensing, or permanent magnet(s) in the case of static magnetic field sensing. The location indicating elements 20 may be placed inside a pouch or chamber 18 that is attached to the flexible joint member 24, or simply attached directly to the distal end portion 16 of the elongated member 12.

When the tip of the medical device encounters an obstacle during insertion, the flexible joint member 24 allows the attached location indicating element 20 to easily bend sideways, at some angle to the direction of travel of the inserted medical device, as shown in FIG. 4. The non-radiographic tracking system 30 (see FIGS. 3-4) will immediately display an abrupt change in direction of the location indicating element 20, indicating its collision with an obstacle.

In one embodiment, the location indicating element 20 consists of one or more permanent magnets inside the closed polymer chamber 18. The distal end of the chamber 18 has a soft, rounded tip, and the proximal end of the chamber is fastened to the distal end portion 16 of the elongated member 12, such as a cardiac catheter, via a flexible polymer joint (i.e., the flexible joint member 24), which has sufficient stiffness to prevent the magnet chamber from bending over under the influence of both gravity and the forces from flowing blood within a patient's vasculature, in any orientation. However, the additional lateral forces on the chamber 18 from encountering an obstacle will cause it to bend sideways.

In one example of operation, consider the placement of a cardiac catheter into a patient's superior vena cava, via the femoral vein. The location of the chamber 18 can be tracked during insertion. If the location indicating element 20 is implemented as a permanent magnet, the static magnetic field may be sensed using an external magnetic field detector (i.e., the detector system 30 of FIGS. 3-4), such as described in the previously referenced patents. If multiple magnets are used, their respective magnetic dipoles are arranged to be additive. The displayed arrow 34 pointing in the catheter's direction of travel as shown on the display 32 in FIGS. 3 and 4. As the catheter elongated member 12 exits the inferior vena cava into the right atrium, the tracking system 30 should display a vertical arrow. If the catheter (i.e., the elongated member 12) then enters the superior vena cava as intended, the location arrow 34 would remain vertical on the display 32. However, if the catheter 12 drifted or turned medially, the chamber 18 would soon encounter the atrial wall and bend sideways. The displayed location arrow 34 would correspondingly turn and point more toward horizontal, as illustrated in FIG. 4, thereby alerting the caregiver to stop pushing the catheter, back it out a short distance, and try again.

From the foregoing it will be appreciated that, although specific embodiments of the invention have been described herein for purposes of illustration, various modifications may be made without deviating from the spirit and scope of the invention. For example, the location indicating element 20 has been described in a number of different embodiments. Those skilled in the art will recognize that other location indicating elements may also be used with the system 10. Accordingly, the present invention is not limited by the specific form of the location indicating element 20.

As another example, the location indicating element 20 may be adjoined to the distal end portion 16 of the elongated member 12 in a variety of different implementations. Different materials and different techniques may be used to flexibly couple the location indicating element 20 to the elongated member 12. Accordingly, the present invention is not limited by any specific method used to flexibly attach the location indicating element 20 to the elongated member 12.

As a further example, the display 32 in FIGS. 3 and 4 may be implemented in a variety of different embodiments. The specific implementation of the display 32 may be dependent on the type of element selected for the location indicating element 20. However, it should be clear that the present invention is not limited by the type of display used to indicate that the elongated member has encountered an obstacle.

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

1. A medical device designed for insertion into a body, the device comprising:
an elongated member having a proximal end and a distal end for insertion into the body; and
a location indicating element flexibly coupled to the distal end of the elongated member and capable of being tracked from a location external to the body wherein an encounter with an obstacle causes a change in the orientation of the location indicating element.

2. The device of claim 1 wherein the location indicating element is flexibly coupled to the elongated member using an elastic polymer.

3. The device of claim 1, further comprising a chamber flexibly coupled to the distal end of the elongated member and having a predetermined orientation with respect to the elongated member wherein the location indicating element is contained within the chamber and wherein an encounter with an obstacle causes a change in the orientation of the chamber.

4. The device of claim 3, further comprising a flexible joint member having first and second ends, the first end being coupled to the distal end of the elongated member and the second end being coupled to the terminal chamber.

5. The device of claim 4 wherein the flexible joint member has sufficient stiffness to maintain the orientation of the chamber under the influence of both gravity and the forces from flowing blood within a patient's vasculature.

6. The device of claim 4 wherein the flexible joint member comprises a solid rubber member.

7. The device of claim 4 wherein the flexible joint member comprises a hollow rubber member.

8. The device of claim 4 wherein the flexible joint member comprises a flexible metal member.

9. The device of claim 4 wherein the flexible joint member comprises an elastic polymer member.

10. The device of claim 1 wherein the location indicating element is a permanent magnet.

11. The device of claim 1 wherein the location indicating element comprises a plurality of permanent magnets oriented with respect to each other such that the respective magnetic fields are additively combined.

12. The device of claim 1 wherein the location indicating element is an electromagnet.

13. The device of claim 1 wherein the location indicating element is a radio frequency coil operating at a predetermined radio frequency.

14. The device of claim 1 wherein the location indicating element is an antenna.

15. The device of claim 1 wherein the location indicating element is a strain relief sensor.

16. The device of claim 1 wherein the elongated member is a selected one of a group comprising a tube, catheter, guidewire, or other insertable medical device.

17. The device of claim 1, further comprising a detector system capable of detecting the location indicating element from a location external to the body, the detector system including a display to indicate an orientation of the location indicating element.

18. A method for detecting an encounter with an obstacle during insertion of a medical device designed for insertion into a body, the method comprising:
selecting an elongated member having a proximal end and a distal end for insertion into the body; and
flexibly coupling a location indicating element to the distal end of the elongated member, the location indicating element being capable of being tracked from a location external to the body wherein an encounter with an obstacle causes a change in the orientation of the location indicating element.

19. The method of claim 18 wherein the location indicating element is flexibly coupled to the elongated member using an elastic polymer member.

20. The method of claim 18, further comprising placing the location indicating element in a chamber flexibly coupled to the distal end of the elongated member to provide the location indicating element within the chamber a predetermined orientation with respect to the elongated member wherein an encounter with an obstacle causes a change in the orientation of the chamber.

21. The method of claim 20 wherein flexibly coupling to the distal end of the elongated member comprises attaching a flexible joint member having first and second ends between the chamber and the elongated member, the first end of the flexible joint member being coupled to the distal end of the elongated member and the second end being coupled to the terminal chamber.

22. The method of claim 21 wherein the flexible joint member has sufficient stiffness to maintain the orientation under the influence of both gravity and the forces from flowing blood within a patient's vasculature.

23. The method of claim 18 wherein the elongated member is a selected one of a group comprising a tube, catheter, guidewire or other medical device.

24. The method of claim 18, further comprising inserting the distal end of the elongated member into the body and monitoring the orientation of the location indicating element wherein an encounter with an obstacle causes a change in the orientation.

25. The method of claim 24 wherein the elongated member is a selected one of a group comprising a tube, catheter, guidewire or other medical device.

26. The method of claim 24, further comprising detecting the location indicating element from a location external to the body and displaying an orientation of the location indicating element.
